(19) United States
(12) Patent Application Publication
(10) Pub. No.: US 2026/0256699 A1
(43) Pub. Date: Sep. 3, 2026

(54) BIOACTIVE MICROSPHERE AND METHOD FOR PREPARATION THEREOF

(71) Applicant: Deepa Vidyadhar Bhajekar, Thane West (IN)

(72) Inventors: Deepa Vidyadhar Bhajekar, Thane West (IN); Subhash Ganesh Ingle, Thane (IN)

(73) Assignee: Deepa Vidyadhar Bhajekar, Thane West (IN)

(21) Appl. No.: 18/713,255

(22) PCT Filed: Nov. 18, 2022
(86) PCT No.: PCT/IN2022/051013

§ 371 Date: May 24, 2024

(30) Foreign Application Priority Data
Nov. 25, 2021 (IN) 202121054594

(51) Int. Cl. A61K 9/16; A61K 31/07; A61K 31/122; A61K 31/355; A61K 31/375; A61K 31/593; A61K 45/06

(52) U.S. Cl. CPC A61K 9/1652; A61K 9/1617; A61K 9/1694; A61K 31/07; A61K 31/122; A61K 31/355; A61K 31/375; A61K 31/593; A61K 45/06

(57) ABSTRACT

Provided herein is a method for preparing a bioactive microsphere. The method involves the blending of a lipophilic phase having a first emulsifier and a first bioactive with an aqueous phase having a second emulsifier and water optionally with second bioactive to form a globular carrier and with the lipophilic phase and the aqueous phase is in the weight ratio range of 1:0.5 to 1:12. The globular carrier is further dispersed in a polymer to obtain a polymeric dispersion, and further the solidification of the polymeric dispersion yields the bioactive microsphere. Also provided are a bioactive microsphere obtained by the method and an enriched food including the bioactive microsphere.

### FIELD OF INVENTION

The subject matter of the present disclosure broadly relates to the field of highly sensitive active agents and enhancement of their stability under conditions of high humidity, water, acidity, light, oxygen, dissolved oxygen, temperature which are normally encountered in food, pharmaceutical and nutraceutical preparations. Particularly, the present disclosure relates to a novel method of preparing a stable bioactive microsphere countering the above-mentioned challenges faced in various matrices retaining the potency of the active over a shelf-life of over 18 months.

### BACKGROUND OF THE INVENTION

Bioactives, when present in small amounts in foods, provide nutritional values and health benefits. The diversity of available bioactives influences different biological properties. Although the bioactives are widely incorporated into food products, the harsh environment of processing, especially in food processes like baking, extruding, retorting, and deep frying, to name a few, create unique problems for the survival of bioactives in finished products. The loss of activity of bioactives usually results in foul odor, discoloration, flavor profile distortion or even in complete loss of potency of bioactives.

When incorporating bioactive such as vitamins, which are essential nutrients in proper functioning of human body as well as animal well-being, into food, another problem arises due to interaction of bioactive with the food base/matrix. Factors like pH, oxygen content, exposure to light and temperature, additives present in the food matrix, etc., can lead to the degradation of the vitamins. Therefore, development of several methods has been explored to protect these sensitive bioactives against decomposition during processing and storage.

Several strategies have been proposed to prevent the degradation of the bioactive in processed foods and beverages. One such preferred method is encapsulation. Encapsulation may be defined as a process to entrap one substance, i.e., active ingredient, within another substance which is a carrier, thereby producing particles with diameters of a few nm to a few mm.

Conventional encapsulation processes are based on making first droplets of the active in gas, liquid or powder form, and these droplets are subsequently surrounded by carrier material in a gas or liquid phase via different physico-chemical processes such as spray-drying, fluid bed coating, spray-chilling/cooling, melt injection, melt extrusion, dripping, emulsification, coacervation, inclusion complexation and liposome entrapment. These methods involve high temperature, use of solvent and other harmful ingredients which might affect the stability of encapsulated actives.

Although, considerable types of encapsulation methods for bioactives are available these days, there are several limitations in terms of poor loading capacity of bioactives, instability, size reduction of encapsulated material, industrial non-feasibility, and prohibitive costs, thereby failing in imparting overall protection from decomposition during processing and storage.

Therefore, there is an emerging need for the development of a low cost and feasible method capable of achieving effective and complete protection of the bioactive against degrading effects of processing, storage, and environmental conditions, along with good stability and loading capacity which allows loading of one or more bioactives.

### SUMMARY OF THE INVENTION

In an aspect of the present disclosure, there is provided a method for preparing a bioactive microsphere, the method comprising: (a) mixing a lipid optionally with a first emulsifier and a first bioactive to obtain a lipophilic phase; (b) mixing a second emulsifier and water optionally with a second bioactive to obtain an aqueous phase; (c) blending the lipophilic phase with the aqueous phase at a temperature in the range of 50 to 95° C. to obtain a globular carrier; (d) dispersing the globular carrier in a polymer in the presence of water to obtain a polymeric dispersion; and (e) solidifying the polymeric dispersion in a gelling bath to obtain the bioactive microsphere, wherein the lipophilic phase and the aqueous phase is in the weight ratio range of 1:0.5 to 1:12; and the bioactive microsphere comprises at least one bioactive selected from the first bioactive, the second bioactive, and combinations thereof.

In a second aspect of the present disclosure, there is provided a bioactive microsphere obtained by the method as disclosed herein, the bioactive microsphere comprises: (a) a lipid; (b) an emulsifier; (c) a polymer; and (d) a bioactive, wherein the bioactive is in the weight range of 0.01 to 40% (w/w); the polymer is in the weight range of 30 to 50% (w/w); the emulsifier is in the weight range of 0.1 to 45% (w/w); and the lipid is in the weight range of 1 to 15% (w/w).

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### BRIEF DESCRIPTION OF THE DRAWINGS

The following drawings form a part of the present specification and are included to further illustrate aspects of the present disclosure. The disclosure may be better understood by reference to the drawings in combination with the detailed description of the specific embodiments presented herein.

FIG. 1 depicts the stability of vitamin D₂-bioactive microsphere in powder form and an enriched food (orange juice) with vitamin D₂-bioactive microsphere at room temperature (average 30° C.) over the span of 9 months, in accordance with an implementation of the present disclosure.

FIG. 2 depicts the stability of an enriched food (orange juice) with vitamin D₃-bioactive microsphere and control encapsulated commercial vitamin D₃ at room temperature (average 30° C.) over the span of 12 months, in accordance with an implementation of the present disclosure.

FIG. 3 depicts the stability of an enriched food (orange juice) with lutein-nutraceutical microsphere and control encapsulated commercial lutein at room temperature (average 30° C.) over the span of 5.5 months, in accordance with an implementation of the present disclosure.

### DETAILED DESCRIPTION OF THE INVENTION

Those skilled in the art will be aware that the present disclosure is subject to variations and modifications other than those specifically described. It is to be understood that the present disclosure includes all such variations and modifications. The disclosure also includes all such steps, features, compositions, and compounds referred to or indicated in this specification, individually or collectively, and any and all combinations of any or more of such steps or features.

### Definitions

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

The term “at least one” is used to mean one or more and thus includes individual components as well as mixtures/combinations.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

The term “including” is used to mean “including but not limited to”. “Including” and “including but not limited to” are used interchangeably.

The term “lipid” used herein refers to a micro-biomolecular compound that is soluble in nonpolar solvents, but insoluble in water. The lipid includes, but not limited to, natural waxes, fats, glycerides, vegetable oils, fatty acids, and fatty acid esters.

The term “bioactive” used herein refers to a compound that is capable of exhibiting biological effect or activity in a living organism. The term bioactive includes, but not limited to, vitamins, anthocyanins, organic extracts, herbal extracts, plant extracts, fruit extracts, rind extract, pomace extract, flower extracts, seed extracts, carotenoids, retinoids, flavors, fragrances, coloring compounds, essential fatty acids, essential amino acids, proteins, active pharmaceutical ingredients (APIs), nutraceutical ingredients, macronutrients, natural or synthetic oils, carbohydrates, proteins, and fats.

The term “vitamin B” used herein refers to at least one water-soluble vitamin from the group of eight chemically distinct compounds that are essential in cell metabolism and synthesis of red blood cells. The term vitamin B includes, but not limited to, vitamin B1, vitamin B2, vitamin B3, vitamin B5, vitamin B6, vitamin B7, vitamin B9, and vitamin B12.

The term “vitamin D” used herein refers to at least one oil-soluble vitamin from the group of five chemically distinct compounds that are essential in intestinal absorption of calcium, magnesium, and phosphate. The term vitamin D includes, but not limited to, vitamin D1, vitamin D2, vitamin D3, vitamin D4, and vitamin D5.

The term “carotenoid” used herein refers to plant pigments that provide fruits and flowers with distinctive red, orange, and yellow colour as well as a number of aromas. Example of carotenoid includes, but not limited to, lutein, zeaxanthin and cryptoxanthin.

The term “emulsifier” used herein refers to a substance capable of stabilizing an emulsion. In the present disclosure, the emulsifier provides stability to the blend of lipophilic and aqueous phases to obtain a mono-dispersed globular carrier. The term emulsifier includes, but not limited to, agar, alginic acid, calcium alginate, sodium alginate, carrageenan, edible gums, dextrin, sorbitol, pectin, sodium pectate, calcium pectate, sodium citrate, sodium phosphates, sodium tartrate, calcium lactate, lecithin, albumin, gelatin, quillaia, modified starches, hydrolysed proteins, glycerides of fatty acids, synthetic lecithin, propylene glycol stearate, propylene glycol alginate, methyl ethyl cellulose, methyl cellulose, sodium carboxy-methyl cellulose, stearyl tartaric acid, esters of monoglycerides and diglycerides of fatty acids, monostearin sodium sulphoacetate, sorbitan esters of fatty acids, poly-oxy-ethylene sorbitan monostearate, poly-oxy-ethylene sorbitan monooleate, sodium stearoyl-2-lactylate, calcium stearoyl-2-lactylate, polyglycerol esters of fatty acids, polyglycerol ester of ricinoleic acid, and esters of wood rosins.

The term “globular carrier” used herein refers to a spherical carrier for a bioactive as defined above, which is substantially homogeneous in size. The globular carrier comprises a homogenous blend of lipophilic phase with the aqueous phase, which is capable of encapsulating the bioactive. In the present disclosure, the globular carrier is in the size range of 50 to 1000 nm.

The term “bioactive microsphere” used herein refers to spherical microparticles of bioactive wherein the bioactive as defined above along with other components such as lipid, emulsifier, and water are dispersed in polymer. In the present disclosure, the bioactive microsphere is in solid or liquid form having size range of 75 to 3000 μm.

The term “HLB value” or “Hydrophilic-lipophilic balance” used herein refers to the value of water or oil solubility for a compound, based on its hydrophilic or lipophilic nature. The HLB values usually range between 0.5 (most lipophilic) and 19.5 (most hydrophilic). In the present disclosure, the lipophilic phase has HLB value in the range of 3 to 8, and the aqueous phase has HLB value in the range of 12 to 20.

The term “additive” used herein refers to a compound which when added to a gelling bath in small amounts, provides solidification of the polymer material used for dispersion of globular carrier via crosslinking the functional groups and/or charges in the polymer. In the present disclosure, the additive added in the gelling bath is selected from a crosslinker, an antisolvent, and a counter polymer, based on the polymer material.

The term “crosslinker” used herein refers to a compound that forms linkage between two adjacent polymeric chains using ionic or covalent bonds. The term crosslinker includes, but not limited to, calcium chloride, calcium sulphate, and calcium carbonate.

The term “antisolvent” used herein refers to a solvent that provided low solubility to a compound. The term antisolvent includes, but not limited to, ethanol, hexane, water, dilute solution of various mineral or organic acids such as hydrochloric acid and acetic acid.

The term “counter polymer” used herein refers to a polymeric compound which carries an opposite charge to the polymer used for the dispersion of bioactive. The term counter polymer includes, but not limited to, chitosan, and poly-L-lysine.

The term “relative humidity” used herein refers to the amount in percentage of water vapour in air that is required for the moisture saturation at a particular temperature. In the present disclosure, the bioactive microsphere is stable at relative humidity in the range of 55% to 80%.

The term “gelling bath” used herein refers to a medium which allows the droplets of polymeric dispersion comprising globular carrier dispersed in aqueous solution of polymer, to solidify in order to obtain a bioactive microsphere.

The term “dripping through syringe needle” used herein refers to the formation of droplets of a polymeric dispersion from the flowing structure formed by syringe needle.

The term “dripping through flow vibration nozzle” used herein refers to the formation of droplets of a polymeric dispersion using fluid flow from the vibrating nozzle driven by a simulated pulsed pressure wave.

The term “spraying” used herein refers to the atomization of polymeric dispersion in form of droplets formed using a nozzle.

The term “emulsion polymerization” used herein refers to the technique in which the polymers or monomers of polymers are first emulsified in the continuous phase using a suitable surfactant before the initiation of the polymerization. The emulsion polymerization can be classified in two categories, aqueous and non-aqueous emulsions, based on the use of either an organic or an aqueous continuous phase.

The term “solidifying” used herein refers to the crosslinking of functional groups and/or charges in the polymer of the polymeric dispersion to form a protective layer around the globular carrier. In the present disclosure, the solidification of polymeric dispersion takes place in a gelling bath to obtain the bioactive microsphere.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a size range of 75 μm to 3000 μm should be interpreted to include not only the explicitly recited limits of 75 μm to 3000 μm, but also to include subranges, such as 100 μm to 2750 μm, 1050 μm to 2000 μm and so forth, as well as individual amounts, including fractional amounts, within the specified ranges, such as 150.5 μm, and 2900 μm, for example.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the disclosure, the preferred methods, and materials are now described. All publications mentioned herein are incorporated herein by reference.

As discussed in the background, the available encapsulation methods face several limitations including poor loading capacity of bioactives, instability, and size reduction of encapsulated material. The industrial non-feasibility and prohibitive costs makes the conventional encapsulation methods for protection of bioactives impractical for commercial use.

In view of the aforementioned shortcomings, it can be understood that an effective, inexpensive, and easy method is required which provides protective layering around the bioactive, and thus, preventing it from the degrading effects of food processing and storage conditions. The present disclosure provides a method for preparation of bioactive microsphere, which comprises mixing a lipid optionally with a first emulsifier and a first bioactive to obtain a lipophilic phase, then mixing a second emulsifier and water optionally with a second bioactive to obtain an aqueous phase, followed by blending the lipophilic phase with the aqueous phase at a temperature in the range of 50 to 95° C. to obtain a globular carrier, which is then dispersed in a polymer in the presence of water to obtain a polymeric dispersion, and finally solidifying the polymeric dispersion in a gelling bath to obtain the bioactive microsphere.

The bioactive microsphere thus obtained from the method of the present disclosure protects the bioactive from deactivating and degrading effects of processing, storage, and environmental conditions, thereby providing stability to the bioactive over longer period of time. The polymer used in the method for preparation of bioactive microsphere forms a protective layer around the bioactive(s). This protective layer is impermeable to the matrices in which they are added, leading to compartmentalization of the bioactive(s) in the food matrix. This compartmentalization prevents the interaction between the highly sensitive bioactives and the food matrices. The polymer also stabilizes the bioactive microsphere of the present disclosure.

The gelling bath used in the method of the present disclosure for solidifying the polymeric dispersion comprises an additive which crosslinks the functional groups and/or charges in the polymer. It is one of the least disruptive methods of microencapsulation, yielding resilient microspheres.

The method of the present disclosure further involves the process of filtration, decantation, washing, or drying. Filtration or decantation process helps to remove the excess additives of the gelling bath from the microsphere. The washing step ensures complete removal of additives from the bioactive microsphere. The drying of bioactive microspheres at low temperature further leads to the enhancement of the stability.

The bioactive microsphere formed from the method of the present disclosure are spherical and mono-dispersed. Mono-dispersed bioactive microspheres lead to the proper handling of the product and avoid segregation and caking. This also leads to the improved sensorial properties. These bioactive microspheres are capable of being loaded with at least one or multiple bioactives at once.

The bioactive microsphere of the present disclosure provides stability of highly sensitive bioactive in aqueous dispersion and improves the resistance against degradation at high temperature during processing, baking, extruding among other processing mechanisms. The present disclosure further provides an enriched food comprising the bioactive microsphere of the present disclosure along with food material.

The present disclosure is not to be limited in scope by the specific embodiments described herein, which are intended for the purposes of exemplification only. Functionally-equivalent products, compositions, and methods are clearly within the scope of the disclosure, as described herein.

In an embodiment of the present disclosure, there is provided a method for preparing a bioactive microsphere, the method comprising: (a) mixing a lipid optionally with a first emulsifier and a first bioactive to obtain a lipophilic phase; (b) mixing a second emulsifier and water optionally with a second bioactive to obtain an aqueous phase; (c) blending the lipophilic phase with the aqueous phase at a temperature in the range of 50 to 95° C. to obtain a globular carrier; (d) dispersing the globular carrier in a polymer in the presence of water to obtain a polymeric dispersion; and (e) solidifying the polymeric dispersion in a gelling bath to obtain the bioactive microsphere, wherein the lipophilic phase and the aqueous phase is in the weight ratio range of 1:0.5 to 1:12; and the bioactive microsphere comprises at least one bioactive selected from the first bioactive, the second bioactive, and combinations thereof.

In an embodiment of the present disclosure, there is provided a method for preparing a bioactive microsphere, the method comprising: (a) mixing a lipid optionally with a first emulsifier and a first bioactive to obtain a lipophilic phase; (b) mixing a second emulsifier and water optionally with a second bioactive to obtain an aqueous phase; (c) blending the lipophilic phase with the aqueous phase at a temperature in the range of 50 to 95° C. to obtain a globular carrier; (d) dispersing the globular carrier in a polymer in the presence of water to obtain a polymeric dispersion; and (e) solidifying the polymeric dispersion in a gelling bath to obtain the bioactive microsphere, wherein the lipophilic phase and the aqueous phase is in the weight ratio range of 1:0.5 to 1:12; and the bioactive microsphere comprises at least one bioactive selected from the first bioactive, the second bioactive, and combinations thereof.

In an embodiment of the present disclosure, there is provided a method for preparing a bioactive microsphere, the method comprising: (a) mixing a lipid with a first emulsifier and a first bioactive to obtain a lipophilic phase; (b) mixing a second emulsifier and water to obtain an aqueous phase; (c) blending the lipophilic phase with the aqueous phase at a temperature in the range of 50 to 95° C. to obtain a globular carrier; (d) dispersing the globular carrier in a polymer in the presence of water to obtain a polymeric dispersion; and (e) solidifying the polymeric dispersion in a gelling bath to obtain the bioactive microsphere, wherein the lipophilic phase and the aqueous phase is in the weight ratio range of 1:0.5 to 1:12; and the bioactive microsphere comprises the first bioactive.

In an embodiment of the present disclosure, there is provided a method for preparing a bioactive microsphere, the method comprising: (a) mixing a lipid to obtain a lipophilic phase; (b) mixing a second emulsifier and water with a second bioactive to obtain an aqueous phase; (c) blending the lipophilic phase with the aqueous phase at a temperature in the range of 50 to 95° C. to obtain a globular carrier; (d) dispersing the globular carrier in a polymer in the presence of water to obtain a polymeric dispersion; and (e) solidifying the polymeric dispersion in a gelling bath to obtain the bioactive microsphere, wherein the lipophilic phase and the aqueous phase is in the weight ratio range of 1:0.5 to 1:12; and the bioactive microsphere comprises the second bioactive.

In an embodiment of the present disclosure, there is provided a method for preparing a bioactive microsphere, the method comprising: (a) mixing a lipid with a first emulsifier and a first bioactive to obtain a lipophilic phase; (b) mixing a second emulsifier and water with a second bioactive to obtain an aqueous phase; (c) blending the lipophilic phase with the aqueous phase at a temperature in the range of 50 to 95° C. to obtain a globular carrier; (d) dispersing the globular carrier in a polymer in the presence of water to obtain a polymeric dispersion; and (e) solidifying the polymeric dispersion in a gelling bath to obtain the bioactive microsphere, wherein the lipophilic phase and the aqueous phase is in the weight ratio range of 1:0.5 to 1:12; and the bioactive microsphere comprises the first bioactive and the second bioactive.

In an embodiment of the present disclosure, there is provided a method for preparing a bioactive microsphere as disclosed herein, wherein solidifying the polymeric dispersion in a gelling bath is carried out by forming droplets of the polymeric dispersion.

In an embodiment of the present disclosure, there is provided a method for preparing a bioactive microsphere as disclosed herein, wherein mixing a lipid optionally with a first emulsifier and a first bioactive is carried out at a temperature in the range of 50 to 125° C.; and mixing a second emulsifier, water and optionally with a second bioactive is carried out at a temperature in the range of 50 to 100° C.

In an embodiment of the present disclosure, there is provided a method for preparing a bioactive microsphere as disclosed herein, wherein the lipophilic phase has HLB value in the range of 3 to 8; and the aqueous phase has HLB value in the range of 12 to 20. In another embodiment of the present disclosure, the lipophilic phase has HLB value in the range of 5 to 8; and the aqueous phase has HLB value in the range of 12 to 18. In yet another embodiment of the present disclosure, the lipophilic phase has a HLB value of 8; and the aqueous phase has a HLB value of 12.

In an embodiment of the present disclosure, there is provided a method for preparing a bioactive microsphere as disclosed herein, wherein the gelling bath comprises an additive selected from a crosslinker, an antisolvent, a counter polymer, or combinations thereof.

In an embodiment of the present disclosure, there is provided a method for preparing a bioactive microsphere as disclosed herein, wherein solidifying the polymeric dispersion in a gelling bath is carried out at a pH in the range of 2 to 8. In another embodiment of the present disclosure, solidifying the polymeric dispersion in a gelling bath is carried out in a pH range of 3 to 6. In yet another embodiment of the present disclosure, solidifying the polymeric dispersion in a gelling bath is carried out at a pH of 3.5.

In an embodiment of the present disclosure, there is provided a method for preparing a bioactive microsphere as disclosed herein, wherein the bioactive microsphere is subjected to a process selected from filtration, decantation, washing, drying, or combinations thereof. In another embodiment of the present disclosure, the bioactive microsphere is subjected to a process selected from filtration, washing, drying, or combinations thereof.

In an embodiment of the present disclosure, there is provided a method for preparing a bioactive microsphere, the method comprising: (a) mixing a lipid optionally with a first emulsifier and a first bioactive at a temperature in the range of 50 to 125° C. to obtain a lipophilic phase having HLB value in the range of 3 to 8; (b) mixing a second emulsifier and water optionally with a second bioactive at a temperature in the range of 50 to 100° C. to obtain an aqueous phase having HLB value in the range of 12 to 20; (c) blending the lipophilic phase with the aqueous phase at a temperature in the range of 50 to 95° C. to obtain a globular carrier; (d) dispersing the globular carrier in a polymer in the presence of water to obtain a polymeric dispersion; and (e) solidifying the polymeric dispersion in a gelling bath comprising an additive selected from a crosslinker, an antisolvent, a counter polymer, or combinations thereof, by forming droplets of the polymeric dispersion at a pH in the range of 2 to 8, to obtain the bioactive microsphere, wherein the lipophilic phase and the aqueous phase is in the weight ratio range of 1:0.5 to 1:12; the bioactive microsphere comprises at least one bioactive selected from the first bioactive, the second bioactive, and combinations thereof; and the bioactive microsphere is subjected to a process selected from filtration, decantation, washing, drying, or combinations thereof.

In an embodiment of the present disclosure, there is provided a method for preparing a bioactive microsphere as disclosed herein, wherein the lipid is selected from natural waxes, fats, glycerides, vegetable oils, fatty acids, fatty acid esters, or combinations thereof. In another embodiment of the present disclosure, the lipid is selected from oleic acid, fats, vegetable oils, fatty acids, fatty acid esters, or combinations thereof. In yet another embodiment of the present disclosure, the lipid is oleic acid.

In an embodiment of the present disclosure, there is provided a method for preparing a bioactive microsphere as disclosed herein, wherein the first bioactive is oil-soluble; the second bioactive is water soluble; and the first bioactive and the second bioactive is independently selected from vitamins, anthocyanins, organic extracts, herbal extracts, plant extracts, fruit extracts, rind extract, pomace extract, flower extracts, seed extracts, carotenoids, retinoids, flavors, fragrances, coloring compounds, essential fatty acids, essential amino acids, proteins, active pharmaceutical ingredients (APIs), macronutrients, natural or synthetic oils, carbohydrates, proteins, fats, or combinations thereof. In another embodiment of the present disclosure, wherein the first bioactive is a vitamin which is oil-soluble. In yet another embodiment of the present disclosure, wherein the second bioactive is a vitamin which is water-soluble.

In an embodiment of the present disclosure, there is provided a method for preparing a bioactive microsphere as disclosed herein, wherein the vitamin is selected from vitamin A, vitamin D, vitamin E, vitamin K, vitamin C, vitamin B, or combinations thereof. In another embodiment of the present disclosure, the vitamin is selected from vitamin A, vitamin D, vitamin B, or combinations thereof. In another embodiment of the present disclosure, the vitamin is vitamin D. In yet another embodiment of the present disclosure, the vitamin D is vitamin D2 or vitamin D3.

In an embodiment of the present disclosure, there is provided a method for preparing a bioactive microsphere as disclosed herein, wherein the vitamin is vitamin B selected from vitamin B1, vitamin B2, vitamin B3, vitamin B5, vitamin B6, vitamin B7, vitamin B9, vitamin B12, or combinations thereof.

In an embodiment of the present disclosure, there is provided a method for preparing a bioactive microsphere, the method comprising: (a) mixing a lipid selected from natural waxes, fats, glycerides, vegetable oils, fatty acids, fatty acid esters, or combinations thereof, optionally with a first emulsifier and a first bioactive to obtain a lipophilic phase; (b) mixing a second emulsifier and water optionally with a second bioactive to obtain an aqueous phase; (c) blending the lipophilic phase with the aqueous phase at a temperature in the range of 50 to 95° C. to obtain a globular carrier; (d) dispersing the globular carrier in a polymer in the presence of water to obtain a polymeric dispersion; and (e) solidifying the polymeric dispersion in a gelling bath to obtain the bioactive microsphere, wherein the lipophilic phase and the aqueous phase is in the weight ratio range of 1:0.5 to 1:12; and the bioactive microsphere comprises at least one bioactive selected from the first bioactive, the second bioactive, and combinations thereof; the first bioactive is oil-soluble; the second bioactive is water soluble; and the first bioactive and the second bioactive is independently selected from vitamin, anthocyanins, organic extracts, herbal extracts, plant extracts, fruit extracts, rind extract, pomace extract, flower extracts, seed extracts, carotenoids, retinoids, flavors, fragrances, coloring compounds, essential fatty acids, essential amino acids, proteins, active pharmaceutical ingredients (APIs), macronutrients, natural or synthetic oils, carbohydrates, proteins, fats, or combinations thereof.

In an embodiment of the present disclosure, there is provided a method for preparing a bioactive microsphere as disclosed herein, wherein the first emulsifier and the second emulsifier is independently selected from agar, alginic acid, calcium alginate, sodium alginate, carrageenan, edible gums, dextrin, sorbitol, pectin, sodium pectate, calcium pectate, sodium citrate, sodium phosphates, sodium tartrate, calcium lactate, lecithin, albumin, gelatin, quillaia, modified starches, hydrolysed proteins, glycerides of fatty acids, synthetic lecithin, propylene glycol stearate, propylene glycol alginate, methyl ethyl cellulose, methyl cellulose, sodium carboxy-methyl cellulose, stearyl tartaric acid, esters of monoglycerides and diglycerides of fatty acids, monostearin sodium sulphoacetate, sorbitan esters of fatty acids, poly-oxy-ethylene sorbitan monostearate, poly-oxy-ethylene sorbitan monooleate, sodium stearoyl-2-lactylate, calcium stearoyl-2-lactylate, polyglycerol esters of fatty acids, polyglycerol ester of ricinoleic acid, esters of wood rosins, or combinations thereof. In another embodiment of the present disclosure, the first emulsifier and the second emulsifier is independently selected from glyceryl monostearate, poly-oxy-ethylene sorbitan monostearate, poly-oxy-ethylene sorbitan monooleate, polyglycerol esters of fatty acids, or combinations thereof. In yet another embodiment of the present disclosure, the first emulsifier is glyceryl monostearate.

In an embodiment of the present disclosure, there is provided a method for preparing a bioactive microsphere as disclosed herein, wherein the polymer is selected from sodium alginate, polypeptide, polysaccharides, agar, agarose, K-carrageenan, alginates, chitosan, cellulose, shellac, methylcellulose, hydroxypropyl cellulose, sodium carboxymethyl cellulose, methyl hydroxypropyl cellulose, collagen, gelatin, egg white, polyacrylamide, polyvinyl alcohol, copoly(styrenemaleic acid), polyethylene glycol (PEG) methacrylate, methoxypolyethylene glycol methacrylate (MPEGMA), PEG dimethacrylate, polyisocyanates, polyurethane, or combinations thereof. In another embodiment of the present disclosure, the polymer is sodium alginate.

In an embodiment of the present disclosure, there is provided a method for preparing a bioactive microsphere, the method comprising: (a) mixing a lipid optionally with a first emulsifier and a first bioactive to obtain a lipophilic phase; (b) mixing a second emulsifier and water optionally with a second bioactive to obtain an aqueous phase; (c) blending the lipophilic phase with the aqueous phase at a temperature in the range of 50 to 95° C. to obtain a globular carrier; (d) dispersing the globular carrier in a polymer selected from sodium alginate, polypeptide, polysaccharides, agar, agarose, K-carrageenan, alginates, chitosan, cellulose, shellac, methylcellulose, hydroxypropyl cellulose, sodium carboxymethyl cellulose, methyl hydroxypropyl cellulose, collagen, gelatin, egg white, polyacrylamide, polyvinyl alcohol, copoly(styrenemaleic acid), polyethylene glycol (PEG) methacrylate, methoxypolyethylene glycol methacrylate (MPEGMA), PEG dimethacrylate, polyisocyanates, polyurethane, or combinations thereof, in the presence of water to obtain a polymeric dispersion; and (e) solidifying the polymeric dispersion in a gelling bath to obtain the bioactive microsphere, wherein the lipophilic phase and the aqueous phase is in the weight ratio range of 1:0.5 to 1:12; and the bioactive microsphere comprises at least one bioactive selected from the first bioactive, the second bioactive, and combinations thereof; and the first emulsifier and the second emulsifier is independently selected from agar, alginic acid, calcium alginate, sodium alginate, carrageenan, edible gums, dextrin, sorbitol, pectin, sodium pectate, calcium pectate, sodium citrate, sodium phosphates, sodium tartrate, calcium lactate, lecithin, albumin, gelatin, quillaia, modified starches, hydrolysed proteins, glycerides of fatty acids, synthetic lecithin, propylene glycol stearate, propylene glycol alginate, methyl ethyl cellulose, methyl cellulose, sodium carboxy-methyl cellulose, stearyl tartaric acid, esters of monoglycerides and diglycerides of fatty acids, monostearin sodium sulphoacetate, sorbitan esters of fatty acids, poly-oxy-ethylene sorbitan monostearate, poly-oxy-ethylene sorbitan monooleate, sodium stearoyl-2-lactylate, calcium stearoyl-2-lactylate, polyglycerol esters of fatty acids, polyglycerol ester of ricinoleic acid, esters of wood rosins, or combinations thereof.

In an embodiment of the present disclosure, there is provided a method for preparing a bioactive microsphere as disclosed herein, wherein the crosslinker is selected from calcium chloride, calcium sulphate, or calcium carbonate; the antisolvent is selected from ethanol, hexane, water, hydrochloric acid, or acetic acid;

and the counter polymer is chitosan, or poly-L-lysine. In another embodiment of the present disclosure, the crosslinker is calcium chloride; and the antisolvent is selected from ethanol, hexane, water, dilute solution of hydrochloric acid and dilute solution of acetic acid.

In an embodiment of the present disclosure, there is provided a method for preparing a bioactive microsphere as disclosed herein, wherein the gelling bath comprises an additive selected from a crosslinker selected from calcium chloride, calcium sulphate, or calcium carbonate; an antisolvent selected from ethanol, hexane, water, hydrochloric acid, or acetic acid; a counter polymer is chitosan, or poly-L-lysine; or combinations thereof.

In an embodiment of the present disclosure, there is provided a method for preparing a bioactive microsphere as disclosed herein, wherein the droplets of the polymeric dispersion is obtained by dripping through syringe needle, flow vibration nozzle, spraying, or emulsion polymerization.

In an embodiment of the present disclosure, there is provided a method for preparing a bioactive microsphere as disclosed herein, wherein solidifying the polymeric dispersion in a gelling bath is carried out by forming droplets of the polymeric dispersion obtained by dripping through syringe needle, flow vibration nozzle, spraying, or emulsion polymerization.

In an embodiment of the present disclosure, there is provided a method for preparing a bioactive microsphere as disclosed herein, wherein the globular carrier is in the size range of 50 to 1000 nm; and the bioactive microsphere is in the size range of 75 to 3000 μm. In another embodiment of the present disclosure, the globular carrier is in the size range of 50 to 300 nm; and the bioactive microsphere is in the size range of 75 to 2000 μm.

In an embodiment of the present disclosure, there is provided a method for preparing a bioactive microsphere as disclosed herein, wherein the globular carrier is spherical, and mono dispersed.

In an embodiment of the present disclosure, there is provided a method for preparing a bioactive microsphere as disclosed herein, wherein the bioactive microsphere is homogenous, spherical, and mono-dispersed.

In an embodiment of the present disclosure, there is provided a method for preparing a bioactive microsphere as disclosed herein, wherein the bioactive microsphere is stable at a temperature in the range of −25 to 30° C.; and at relative humidity in the range of 55% to 80%.

In an embodiment of the present disclosure, there is provided a method for preparing a bioactive microsphere as disclosed herein, wherein the bioactive microsphere is stable at a processing temperature in the range of −25 to 260° C.

In an embodiment of the present disclosure, there is provided a method for preparing a bioactive microsphere, the method comprising: (a) mixing a lipid optionally with a first emulsifier and a first bioactive to obtain a lipophilic phase; (b) mixing a second emulsifier and water optionally with a second bioactive to obtain an aqueous phase; (c) blending the lipophilic phase with the aqueous phase at a temperature in the range of 50 to 95° C. to obtain a globular carrier in the size range of 50 to 1000 nm; (d) dispersing the globular carrier in a polymer in the presence of water to obtain a polymeric dispersion; and (e) solidifying the polymeric dispersion in a gelling bath to obtain the bioactive microsphere in the size range of 75 to 3000 μm, wherein the lipophilic phase and the aqueous phase is in the weight ratio range of 1:0.5 to 1:12; and the bioactive microsphere comprises at least one bioactive selected from the first bioactive, the second bioactive, and combinations thereof; the bioactive microsphere is stable at a temperature in the range of −25 to 30° C.; and at relative humidity in the range of 55% to 80%.

In an embodiment of the present disclosure, there is provided a method for preparing a bioactive microsphere, the method comprising: (a) mixing a lipid optionally with a first emulsifier and a first bioactive to obtain a lipophilic phase; (b) mixing a second emulsifier and water optionally with a second bioactive to obtain an aqueous phase; (c) blending the lipophilic phase with the aqueous phase at a temperature in the range of 50 to 95° C. to obtain a globular carrier in the size range of 50 to 1000 nm; (d) dispersing the globular carrier in a polymer in the presence of water to obtain a polymeric dispersion; and (e) solidifying the polymeric dispersion in a gelling bath to obtain the bioactive microsphere in the size range of 75 to 3000 μm, wherein the lipophilic phase and the aqueous phase is in the weight ratio range of 1:0.5 to 1:12; and the bioactive microsphere comprises at least one bioactive selected from the first bioactive, the second bioactive, and combinations thereof; the bioactive microsphere is stable at a processing temperature in the range of −25 to 260° C.

In an embodiment of the present disclosure, there is provided a bioactive microsphere obtained by the method as disclosed herein, the bioactive microsphere comprises: (a) a lipid; (b) an emulsifier; (c) a polymer; and (d) a bioactive, wherein the bioactive is in the weight range of 0.01 to 40% (w/w); the polymer is in the weight range of 30 to 50% (w/w); the emulsifier is the weight range of 0.1 to 45% (w/w); and the lipid is in the weight range of 1 to 15% (w/w). In another embodiment of the present disclosure, the emulsifier is the weight range of 0.2% to 10%. In yet another embodiment of the present disclosure, the emulsifier is the weight range of 0.25% to 2%. In another embodiment of the present disclosure, the bioactive is in the weight range of 0.01 to 20%. In yet another embodiment of the present disclosure, the bioactive is in the weight range of 0.01 to 15%.

In an embodiment of the present disclosure, there is provided a bioactive microsphere obtained by the method comprising: (i) mixing a lipid optionally with a first emulsifier and a first bioactive to obtain a lipophilic phase; (ii) mixing a second emulsifier and water optionally with a second bioactive to obtain an aqueous phase; (iii) blending the lipophilic phase with the aqueous phase at a temperature in the range of 50 to 95° C. to obtain a globular carrier; (iv) dispersing the globular carrier in a polymer in the presence of water to obtain a polymeric dispersion; and (v) solidifying the polymeric dispersion in a gelling bath to obtain the bioactive microsphere, wherein the lipophilic phase and the aqueous phase is in the weight ratio range of 1:0.5 to 1:12; and the bioactive microsphere comprises at least one bioactive selected from the first bioactive, the second bioactive, and combinations thereof, the bioactive microsphere comprises: (a) a lipid; (b) an emulsifier; (c) a polymer; and (d) a bioactive, wherein the bioactive is in the weight range of 0.01 to 40% (w/w); the polymer is in the weight range of 30 to 50% (w/w); the emulsifier is the weight range of 0.1 to 45% (w/w); and the lipid is in the weight range of 1 to 15% (w/w).

In an embodiment of the present disclosure, there is provided a bioactive microsphere as disclosed herein, wherein the microsphere further comprises water, and a crosslinker, and the microsphere is in solid form, or in liquid form.

In an embodiment of the present disclosure, there is provided a bioactive microsphere as disclosed herein, wherein the microspheres keep bioactives stable in a pH range of 2.0 to 8.0; and for a period of 5 months to 18 months.

In an embodiment of the present disclosure, there is provided a bioactive microsphere obtained by the method as disclosed herein, the bioactive microsphere comprises: (a) a lipid; (b) an emulsifier; (c) a polymer; and (d) a bioactive, wherein the bioactive is in the weight ratio range of 0.01 to 40% (w/w); the polymer is in the weight ratio range of 30 to 50% (w/w); the emulsifier is the weight ratio range of 0.1 to 45% (w/w); and the lipid is in the weight ratio range of 1 to 15% (w/w); the microsphere further comprises water; the microsphere further comprises a crosslinker; and the microsphere is in solid form, or in liquid form.

In an embodiment of the present disclosure, there is provided a bioactive microsphere, the bioactive microsphere comprises: (a) a lipid selected from natural waxes, fats, glycerides, vegetable oils, fatty acids, fatty acid esters, or combinations thereof; (b) an emulsifier selected from agar, alginic acid, calcium alginate, sodium alginate, carrageenan, edible gums, dextrin, sorbitol, pectin, sodium pectate, calcium pectate, sodium citrate, sodium phosphates, sodium tartrate, calcium lactate, lecithin, albumin, gelatin, quillaia, modified starches, hydrolysed proteins, glycerides of fatty acids, synthetic lecithin, propylene glycol stearate, propylene glycol alginate, methyl ethyl cellulose, methyl cellulose, sodium carboxy-methyl cellulose, stearyl tartaric acid, esters of monoglycerides and diglycerides of fatty acids, monostearin sodium sulphoacetate, sorbitan esters of fatty acids, poly-oxy-ethylene sorbitan monostearate, poly-oxy-ethylene sorbitan monooleate, sodium stearoyl-2-lactylate, calcium stearoyl-2-lactylate, polyglycerol esters of fatty acids, polyglycerol ester of ricinoleic acid, esters of wood rosins, or combinations thereof; (c) a polymer selected from sodium alginate, polypeptide, polysaccharides, agar, agarose, K-carrageenan, alginates, chitosan, cellulose, shellac, methylcellulose, hydroxypropyl cellulose, sodium carboxymethyl cellulose, methyl hydroxypropyl cellulose, collagen, gelatin, egg white, polyacrylamide, polyvinyl alcohol, copoly(styrenemaleic acid), polyethylene glycol (PEG) methacrylate, methoxypolyethylene glycol methacrylate (MPEGMA), PEG dimethacrylate, polyisocyanates, polyurethane, or combinations thereof; and (d) a bioactive selected from vitamins, organic extracts, herbal extracts, plant extracts, fruit extracts, rind extract, pomace extract, flower extracts, seed extracts, carotenoids, retinoids, flavors, fragrances, coloring compounds, essential nutraceuticals ingredients, macronutrients, natural or synthetic oils, carbohydrates, proteins, fats, or combinations thereof, wherein the bioactive is in the weight ratio range of 0.01 to 40% (w/w); the polymer is in the weight ratio range of 30 to 50% (w/w); the emulsifier is the weight ratio range of 0.1 to 45% (w/w); and the lipid is in the weight ratio range of 1 to 15% (w/w); and the microsphere is in solid form, or in liquid form.

In an embodiment of the present disclosure, there is provided a bioactive microsphere obtained by the method as disclosed herein, the bioactive microsphere comprises: (a) a lipid; (b) an emulsifier; (c) a polymer; and (d) a bioactive, wherein the bioactive is in the weight ratio range of 0.01 to 40% (w/w); the polymer is in the weight ratio range of 30 to 50% (w/w); the emulsifier is the weight ratio range of 0.1 to 45% (w/w); and the lipid is in the weight ratio range of 1 to 15% (w/w)); and wherein the microsphere further comprises a crosslinker selected from calcium chloride, calcium sulphate, calcium carbonate, or combinations thereof.

In an embodiment of the present disclosure, there is provided a bioactive microsphere comprising: (a) a lipid; (b) an emulsifier; (c) a polymer; and (d) a bioactive, wherein the bioactive is in the weight ratio range of 0.01 to 40% (w/w); the polymer is in the weight ratio range of 30 to 50% (w/w); the emulsifier is the weight ratio range of 0.1 to 45% (w/w); and the lipid is in the weight ratio range of 1 to 15% (w/w).

In an embodiment of the present disclosure, there is provided a bioactive microsphere comprising: (a) a lipid; (b) an emulsifier; (c) a polymer; and (d) a bioactive, wherein the bioactive is in the weight ratio range of 0.01 to 40% (w/w); the polymer is in the weight ratio range of 30 to 50% (w/w); the emulsifier is the weight ratio range of 0.1 to 45% (w/w); and the lipid is in the weight ratio range of 1 to 15% (w/w); the microsphere further comprises water; the microsphere further comprises a crosslinker; and the microsphere is in solid form, or in liquid form.

In an embodiment of the present disclosure, there is provided a bioactive microsphere comprising: (a) a lipid; (b) an emulsifier; (c) a polymer; and (d) a bioactive, wherein the bioactive is in the weight ratio range of 0.01 to 40% (w/w); the polymer is in the weight ratio range of 30 to 50% (w/w); the emulsifier is the weight ratio range of 0.1 to 45% (w/w); and the lipid is in the weight ratio range of 1 to 15% (w/w); and the microsphere further comprises a crosslinker selected from calcium chloride, calcium sulphate, calcium carbonate, or combinations thereof.

In an embodiment of the present disclosure, there is provided a bioactive microsphere, the bioactive microsphere comprises: (a) a lipid selected from natural waxes, fats, glycerides, vegetable oils, fatty acids, fatty acid esters, or combinations thereof; (b) an emulsifier selected from agar, alginic acid, calcium alginate, sodium alginate, carrageenan, edible gums, dextrin, sorbitol, pectin, sodium pectate, calcium pectate, sodium citrate, sodium phosphates, sodium tartrate, calcium lactate, lecithin, albumin, gelatin, quillaia, modified starches, hydrolysed proteins, glycerides of fatty acids, synthetic lecithin, propylene glycol stearate, propylene glycol alginate, methyl ethyl cellulose, methyl cellulose, sodium carboxy-methyl cellulose, stearyl tartaric acid, esters of monoglycerides and diglycerides of fatty acids, monostearin sodium sulphoacetate, sorbitan esters of fatty acids, poly-oxy-ethylene sorbitan monostearate, poly-oxy-ethylene sorbitan monooleate, sodium stearoyl-2-lactylate, calcium stearoyl-2-lactylate, polyglycerol esters of fatty acids, polyglycerol ester of ricinoleic acid, esters of wood rosins, or combinations thereof; (c) a polymer selected from sodium alginate, polypeptide, polysaccharides, agar, agarose, K-carrageenan, alginates, chitosan, cellulose, shellac, methylcellulose, hydroxypropyl cellulose, sodium carboxymethyl cellulose, methyl hydroxypropyl cellulose, collagen, gelatin, egg white, polyacrylamide, polyvinyl alcohol, copoly(styrenemaleic acid), polyethylene glycol (PEG) methacrylate, methoxypolyethylene glycol methacrylate (MPEGMA), PEG dimethacrylate, polyisocyanates, polyurethane, or combinations thereof; and (d) a bioactive selected from vitamins, organic extracts, herbal extracts, plant extracts, fruit extracts, rind extract, pomace extract, flower extracts, seed extracts, carotenoids, retinoids, flavors, fragrances, coloring compounds, essential nutraceuticals ingredients, macronutrients, natural or synthetic oils, carbohydrates, proteins, fats, or combinations thereof, wherein the bioactive is in the weight ratio range of 0.01 to 40% (w/w); the polymer is in the weight ratio range of 30 to 50% (w/w); the emulsifier is the weight ratio range of 0.1 to 45% (w/w); and the lipid is in the weight ratio range of 1 to 15% (w/w).

In an embodiment of the present disclosure, there is provided a bioactive microsphere as disclosed herein, wherein the microsphere enhances the stability of the bioactives encompassing many others under conditions of high humidity, water, acidity, light, oxygen, dissolved oxygen, temperature, other additives which are normally encountered in food, pharmaceutical and nutraceutical preparations.

In an embodiment of the present disclosure, there is provided an enriched food comprising: (a) a food; and (b) the bioactive microsphere as disclosed herein, wherein the bioactive microsphere is in the weight ratio range of 0.000001 to 10% (w/w).

In an embodiment of the present disclosure, there is provided an enriched food comprising: (a) a food; and (b) the bioactive microsphere comprising: i) a lipid; (ii) an emulsifier; (iii) a polymer; and (iv) a bioactive, wherein the bioactive is in the weight ratio range of 0.01 to 40% (w/w); the polymer is in the weight ratio range of 30 to 50% (w/w); the emulsifier is the weight ratio range of 0.1 to 45% (w/w); and the lipid is in the weight ratio range of 1 to 15% (w/w), and the bioactive microsphere is in the weight ratio range of 0.000001 to 10% (w/w).

In an embodiment of the present disclosure, there is provided an enriched food as disclosed herein, wherein the food is selected from milk, non-dairy milk, cream, curd, paneer, cheese, ice cream, kulfi, softy, milk powder, infant food, butter, ghee, shrikhand, sweets, yoghurt, oils, margarine, fat spreads, fruit and vegetable juice, juice concentrates, squashes, crushes, fruit syrups, fruit, sharbats, barley water, murabba, ketchup, sauce, carbonated fruit beverages, jam, jelly, marmalades, chutney, pickles, atta flour, maida, semolina, besan, edible powders and mixes, pasta, bread, meat products including meat preparations, fish products, sweets, confectionery, honey, chocolate, spices, spice powder, spice extracts, salt, tea, coffee, other food products including powders, solids, liquids, pastes, gels, doughs, curries, curry powders, ready to cook food, ready to eat food, or combinations thereof. In another embodiment of the present disclosure, wherein the food is in different form such as liquids, solids, pastes, creams, or emulsions. In yet another embodiment of the present disclosure, the microspheres keep bioactives essentially Vitamin D2, Vitamin D3, Vitamin A, Vitamin E, Vitamin K stable even when placed in strongly acidic beverage such as orange juice.

Although the subject matter has been described in considerable detail with reference to certain examples and implementations thereof, other implementations are possible.

### EXAMPLES

The disclosure will now be illustrated with working examples, which is intended to illustrate the working of disclosure and not intended to take restrictively to imply any limitations on the scope of the present disclosure. Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices, and materials are described herein. It is to be understood that this disclosure is not limited to particular methods, and experimental conditions described, as such methods and conditions may apply.

The forthcoming examples explain how the present disclosure provides a method for preparing a bioactive microsphere. The present disclosure also provides an enriched food comprising the bioactive microsphere as prepared in the Examples below. The examples demonstrate the stability of bioactive in the bioactive microsphere of the present disclosure when subjected to processing environment, for instance, boiling.

### Materials and Methods

For the purpose of the present disclosure, following raw materials with the specified grades/brands were used.
a) Oleic acid, 99%;
b) Glyceryl monostearate, 99%;
c) Sodium alginate, 99%;

### Example 1

### Method for Preparation of a Vitamin D2 Loaded Bioactive Microsphere

In an example, the method of the present disclosure involves preparation of bioactive microsphere with Vitamin D2 (oil soluble) as the first bioactive. The lipid used in the method was oleic acid. The first and second emulsifiers used were glyceryl monostearate and poly-oxy-ethylene sorbitan monooleate, respectively. Sodium alginate was used as the polymer for forming a protective layer around the globular carrier.

0.9 g of oleic acid was mixed with 3 g of glyceryl monostearate and 0.1 g of Vitamin D2 at 70° C. to obtain a lipophilic phase. Then, in a separate beaker, 2 g of poly-oxy-ethylene sorbitan monostearate was mixed with water at 70° C. to obtain an aqueous phase. The two phases, i.e., lipophilic, and aqueous phases, were then blended together such that the weight ratio of lipophilic phase and the aqueous phase is 1:12. The blending step was performed at the temperature of 70° C. under rapid stirring emulsion, to form vitamin D2 loaded globular carrier. This globular carrier was dispersed in the aqueous solution of 1.5% sodium alginate solution to obtain a polymeric dispersion. The alginate provided stability to the microspheres prepared from the method by forming a protective layer around the globular carrier. The droplets of the polymeric dispersion were then added dropwise via syringe needle in a gelling bath comprising CaCl₂) as crosslinker, at a pH of 3.5. The pH of the gelling bath can be adjusted by using any mineral or organic acid, such as hydrochloric acid, or acetic acid, or by using sodium hydroxide, or sodium bicarbonate.

The final step of solidifying the polymeric dispersion allowed the formation of vitamin D2 loaded bioactive microsphere. The microspheres were then separated by filtration, followed by washing to remove any traces of CaCl₂, and further subjected to drying at a low temperature of 65° C. The drying step at low temperatures led to the enhanced stability of the microspheres.

The obtained microspheres were spherical and mono-dispersed. The average diameter of these microspheres was found to be less than 2000 μm.

### Example 2

### Stability Characterization of the Vitamin D2 Loaded Bioactive Microsphere in Milk

In an example, the effect of bioactive microsphere of the present disclosure on the stability of bioactive, when subjected to the deteriorating effect of processing parameter such as heating, was studied. The stability of vitamin D2 was determined by heating the milk containing the bioactive microsphere as prepared in Example 1, and the milk containing pure vitamin D2, i.e., control vitamin D2.

For the purpose of this example, both the vitamin D2 containing milk were boiled twice, and the amount of vitamin D2 was determined after second boiling, as shown in Table 1. The difference in the amount of vitamin D2 in the milk before boiling, and after the second boiling, illustrated the deteriorating effect of heat on the vitamin D2.

TABLE 1
Relative assay of Vitamin D₂ in milk

| | % Relative assay | | |
|---|---|---|---|
| | Before | After second | % |
| Test samples | boiling (%) | boiling (%) | Degradation |
| Vitamin D₂-bioactive | 100 | 95 | 5 |
| microsphere | | | |
| Control vitamin D₂ | 100 | 35 | 65 |

From the above Table 1, it was observed that the bioactive microsphere of the present disclosure improved the stability of vitamin D2 in milk even after boiling two times, whereas control vitamin D2 deactivated on boiling making it unavailable in the full quantity as intended for the consumer.

### Example 3

### Stability Characterization of the Vitamin D2 Loaded Bioactive Microsphere in Juice

In an example, the effect of bioactive microsphere of the present disclosure on the stability of bioactive, when subjected to the deteriorating effect of matrix such as pH, dissolved oxygen and water was studied.

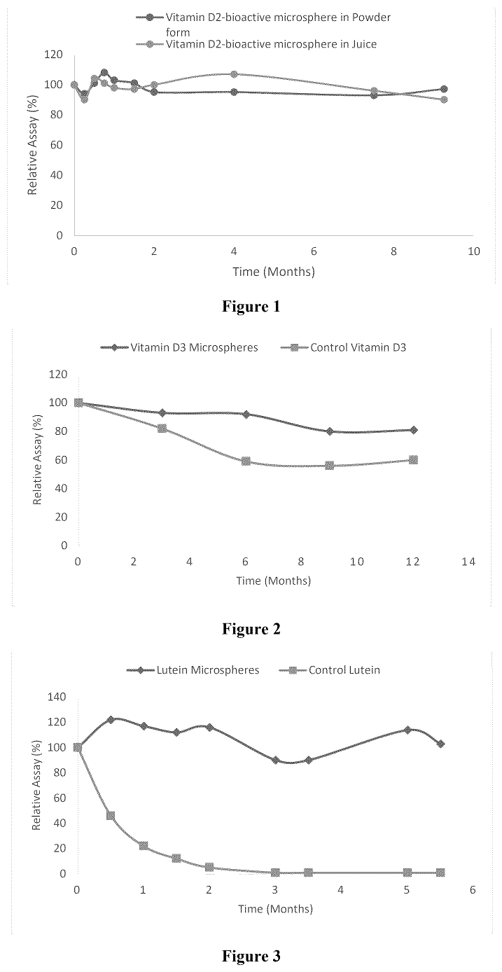

The stability of vitamin D2 using the bioactive microsphere of the present disclosure was confirmed by the stability data as shown in FIG. 1. The stability data illustrated that the bioactive microsphere (Powder) of the present disclosure were stable at room temperature (RT) and as well as in acidic beverage (Juice), which clearly indicated the prevention of bioactive (vitamin D2) from degradation due to oxidation, temperature, pH, and high-water content.

### Example 4

### Method for Preparation of a Vitamin D3 Loaded Bioactive Microsphere

The procedure of Example 1 is followed, replacing 0.1 g of Vitamin D2 with 0.1 g of Vitamin D3.

The obtained microspheres were spherical and mono-dispersed. The average diameter of these microspheres was found to be less than 2000 μm.

### Example 5

### Stability Characterization of the Vitamin D3 Loaded Bioactive Microsphere in Juice

In an example, the effect of bioactive microsphere of the present disclosure on the stability of bioactive, when subjected to the deteriorating effect of matrix parameters such as low pH, was studied. The stability of vitamin D3 over the period of time was determined by keeping the juice containing the bioactive microsphere as prepared in Example 3, and the juice containing commercial vitamin D3, i.e., control vitamin D3 for the period of 1 year at room temperature.

TABLE 2
Relative assay of Vitamin D₃ in juice

| | % Relative assay | | % |
|---|---|---|---|
| Test samples | On 0 day | After 1 year | Degradation |
| Vitamin D₃- | 100 | 81 | 19 |
| bioactive | | | |
| microsphere | | | |
| Control vitamin D₃ | 100 | 60 | 40 |

From the Table 2 and FIG. 2, it was observed that the bioactive microsphere of the present disclosure improved the stability of vitamin D3 in juice even after 1 year, whereas control vitamin D3 reduced by 40% making it unavailable in the full quantity as intended for the consumer.

The stability data illustrated that the bioactive microsphere of the present disclosure, were stable at room temperature (RT) and as well as in acidic beverage, which clearly indicated the prevention of bioactive (vitamin D3) from degradation due to oxidation, temperature, pH, and high water content.

### Example 6

### Method for Preparation of a Lutein Loaded Nutraceutical Microsphere

The procedure of Example 1 is followed, replacing 0.1 g of Vitamin D2 with 0.2 g of Lutein.

The obtained microspheres were spherical and mono-dispersed. The average diameter of these microspheres was found to be less than 2000 μm.

### Example 7

### Stability Characterization of the Lutein Loaded Nutraceutical Microsphere in Juice

In an example, the effect of bioactive microsphere of the present disclosure on the stability of bioactive, when subjected to the deteriorating effect of matrix parameters such as low pH, was studied. The stability of Lutein over the period of time was determined by keeping the juice containing the neutraceutical microsphere as prepared in Example 6, and the juice containing commercial Lutein, i.e., control Lutein for the period of 5.5 months at room temperature.

TABLE 3
% Relative assay of Lutein in juice

| | % Relative assay | | % |
|---|---|---|---|
| Test samples | On 0 day | After 5.5 months | Degradation |
| Lutein-nutraceutical | 100 | 103 | 0 |
| microsphere | | | |
| Control Lutein | 100 | 1 | 99 |

From the Table 3 and FIG. 3, it was observed that the neutraceutical microsphere of the present disclosure improved the stability of Lutein in juice even after 1 year, whereas control Lutein reduced drastically making it unavailable in the full quantity as intended for the consumer.

The stability data illustrated that the bioactive microsphere of the present disclosure, were stable in acidic beverage at room temperature, which clearly indicated the prevention of neutraceutical (Lutein) from degradation due to oxidation, temperature, pH and high water content.

### Example 8

### Method for Preparation of a Vitamin D2 Loaded Bioactive Microsphere with Different Amount of Sodium Alginate

The procedure of Example 1 is followed, replacing 1.5% Sodium alginate solution with different concentrations of sodium alginate, viz., 0.25%, 0.5%, 1%, 1.5% & 2% respectively.

The obtained microspheres were spherical and mono-dispersed. The average diameter of these microspheres was found to be less than 2000 μm.

### Example 9

### Stability Characterization of the Vitamin D2 Loaded Bioactive Microsphere with Different Amount of Sodium Alginate in Juice

In an example, the effect of different concentrations of sodium alginate on the stability of bioactive, when subjected to the deteriorating effect of matrix parameters such as low pH, was studied. The stability of vitamin D2 over the period of time was determined by keeping the juice containing the bioactive microspheres as prepared in Example 8 for the period of 1 year at room temperature.

TABLE 4
% Relative assay of vitamin D₂ in juice

| Sodium alginate | % Relative assay | | % |
|---|---|---|---|
| concentration (%) | On 0 day | After 1 year | Degradation |
| 0.25 | 100 | 36 | 64 |
| 0.5 | 100 | 67 | 33 |
| 1 | 100 | 81 | 19 |
| 1.5 | 100 | 95 | 5 |
| 2 | 100 | 96 | 4 |

From the above Table 4, it was observed that the bioactive microsphere of the present disclosure with higher concentration of sodium alginate improved the stability of vitamin D2 in juice even after 1 year, whereas the bioactive microsphere of the present disclosure with lower concentration of sodium alginate lead to the reduced stability of Vitamin D2 making it unavailable in the full quantity as intended for the consumer.

### Advantages of the Present Disclosure

The present disclosure discloses a method for preparing a bioactive microsphere. The present disclosure also discloses a bioactive microsphere obtained by the method as disclosed herein, and an enriched food comprising said bioactive microsphere.

The method of the present disclosure is a low cost and facile way for preparing bioactive microspheres and provides encapsulation of highly sensitive bioactives during conditions of processing, storage, and environmental effects, thereby preventing the degradation of these bioactives. The lipophilic phase and the aqueous phase allow mixing of oil soluble and water soluble bioactives, respectively. The method involves a drying step at low temperatures which enhances stability of the microspheres. The use of first and/or second emulsifier in the method of the present disclosure results in stable globular carrier, which is spherical and mono-dispersed. The mono-dispersed globular carrier stabilized using the emulsifier results in the mono-dispersed and homogeneous bioactive microsphere of the present disclosure.

The bioactive microsphere is capable of being dispersible in water and oil-soluble food, thereby providing nutritious value to the food by incorporating the bioactive microsphere of the present disclosure. This allows the application of the bioactive microsphere in multidisciplinary end-uses for food, nutraceutical, and pharma industry. The bioactive microsphere of the present disclosure also provides improved stability to bioactives. These microspheres are capable of being loaded with at least one or multiple bioactives at once. The microspheres enhance the stability of mentioned bioactives encompassing many others under conditions of high humidity, water, acidity, light, oxygen, dissolved oxygen, temperature, other additives which are normally encountered in food, pharmaceutical and nutraceutical preparations.

Further, the encapsulation of bioactive using the bioactive microsphere of the present disclosure provides effective resistance against the degradation effect at high temperature during processing, baking, extruding among other processing mechanisms.

1. A method for preparing a bioactive microsphere, the method comprising:
a) mixing a lipid optionally with a first emulsifier and a first bioactive to obtain a lipophilic phase;
b) mixing a second emulsifier and water optionally with a second bioactive to obtain an aqueous phase;
c) blending the lipophilic phase with the aqueous phase at a temperature in the range of 50 to 95° C. to obtain a globular carrier;
d) dispersing the globular carrier in a polymer in the presence of water to obtain a polymeric dispersion; and
e) solidifying the polymeric dispersion in a gelling bath to obtain the bioactive microsphere,
wherein the lipophilic phase and the aqueous phase is in the weight ratio range of 1:0.5 to 1:12; and the bioactive microsphere comprises at least one bioactive selected from the first bioactive, the second bioactive, and combinations thereof.

2. The method as claimed in claim 1, wherein solidifying the polymeric dispersion in a gelling bath is carried out by forming droplets of the polymeric dispersion.

3. The method as claimed in claim 1, wherein mixing a lipid optionally with a first emulsifier and a first bioactive is carried out at a temperature in the range of 50 to 125° C.; and mixing a second emulsifier, water and optionally with a second bioactive is carried out at a temperature in the range of 50 to 100° C.

4. The method as claimed in claim 1, wherein the lipophilic phase has HLB value in the range of 3 to 8; and the aqueous phase has HLB value in the range of 12 to 20.

5. The method as claimed in claim 1, wherein the gelling bath comprises an additive selected from a crosslinker, an antisolvent, a counter polymer, or combinations thereof.

6. The method as claimed in claim 1, wherein solidifying the polymeric dispersion in a gelling bath is carried out at a pH in the range of 2 to 8.

7. The method as claimed in claim 1, wherein the bioactive microsphere is subjected to a process selected from filtration, decantation, washing, drying, or combinations thereof.

8. The method as claimed in claim 1, wherein the lipid is selected from natural waxes, fats, glycerides, vegetable oils, fatty acids, fatty acid esters, or combinations thereof.

9. The method as claimed in claim 1, wherein the first bioactive is oil-soluble; the second bioactive is water soluble; and the first bioactive and the second bioactive is are independently selected from vitamins, anthocyanins, organic extracts, herbal extracts, plant extracts, fruit extracts, rind extract, pomace extract, flower extracts, seed extracts, carotenoids, retinoids, flavors, fragrances, coloring compounds, essential fatty acids, essential amino acids, proteins, active pharmaceutical ingredients (APIs), nutraceuticals ingredients, macronutrients, natural or synthetic oils, carbohydrates, proteins, fats, or combinations thereof, and wherein the vitamin is selected from vitamin A, vitamin D, vitamin E, vitamin K, vitamin C, vitamin B, or combination thereof.

10. (canceled)

11. The method as claimed in claim 1, wherein the first emulsifier and the second emulsifier is independently selected from agar, alginic acid, calcium alginate, sodium alginate, carrageenan, edible gums, dextrin, sorbitol, pectin, sodium pectate, calcium pectate, sodium citrate, sodium phosphates, sodium tartrate, calcium lactate, lecithin, albumin, gelatin, quillaia, modified starches, hydrolysed proteins, glycerides of fatty acids, synthetic lecithin, propylene glycol stearate, propylene glycol alginate, methyl ethyl cellulose, methyl cellulose, sodium carboxy-methyl cellulose, stearyl tartaric acid, esters of monoglycerides and diglycerides of fatty acids, monostearin sodium sulphoacetate, sorbitan esters of fatty acids, poly-oxy-ethylene sorbitan monostearate, poly-oxy-ethylene sorbitan monooleate, sodium stearoyl-2-lactylate, calcium stearoyl-2-lactylate, polyglycerol esters of fatty acids, polyglycerol ester of ricinoleic acid, esters of wood rosins, or combinations thereof.

12. The method as claimed in claim 1, wherein the polymer is selected from sodium alginate, polypeptide, polysaccharides, agar, agarose, k-carrageenan, alginates, chitosan, cellulose, shellac, methylcellulose, hydroxypropyl cellulose, sodium carboxymethyl cellulose, methyl hydroxypropyl cellulose, collagen, gelatin, egg white, polyacrylamide, polyvinyl alcohol, copoly(styrenemaleic acid), polyethylene glycol (PEG) methacrylate, methoxypolyethylene glycol methacrylate (MPEGMA), PEG dimethacrylate, polyisocyanates, polyurethane, or combinations thereof.

13. The method as claimed in claim 5, wherein the crosslinker is selected from calcium chloride, calcium sulphate, or calcium carbonate; the antisolvent is selected from ethanol, hexane, water, hydrochloric acid, or acetic acid; and the counter polymer is chitosan, or poly-L-lysine.

14. The method as claimed in claim 1, wherein the droplet of the polymeric dispersion is obtained by dripping through syringe needle, flow vibration nozzle, spraying, or emulsion polymerization.

15. The method as claimed in claim 1, wherein the globular carrier is in the size range of 50 to 1000 nm; and the bioactive microsphere is in the size range of 75 to 3000 μm.

16. The method as claimed in claim 1, wherein the bioactive microsphere is stable at a storage temperature in the range of −25 to 40° C.; and at relative humidity in the range of 55% to 80%.

17. The method as claimed in claim 1, wherein the bioactive microsphere is stable at a processing temperature in the range of −25 to 260° C.

18. A bioactive microsphere obtained by the method as claimed in claim 1, the bioactive microsphere comprises:
a) a lipid;
b) an emulsifier;
c) a polymer; and
d) a bioactive,
wherein the bioactive is in the weight range of 0.01 to 40% (w/w); the polymer is in the weight range of 30 to 50% (w/w); the emulsifier is the weight range of 0.1 to 45% (w/w); and the lipid is in the weight range of 1 to 15% (w/w).

19. The microsphere as claimed in claim 18, wherein the microsphere comprises water, and a crosslinker; and the microsphere is in solid form, or in liquid form.

20. The microsphere as claimed in claim 18, wherein the microsphere is stable at a storage temperature in the range of −25 to 40° C. and is stable at a processing temperature in the range of −25 to 260° C.

21. The microsphere as claimed in claim 18, wherein the microsphere keep bioactives in a pH range of 2 to 8; and for a period of 5 months to 18 months.
